(19) United States
(12) Patent Application Publication
(10) Pub. No.: US 2021/0380910 A1
(43) Pub. Date: Dec. 9, 2021

(54) CELL COUNTING AND CULTURE INTERPRETATION METHOD AND APPLICATION THEREOF

(71) Applicant: Schweitzer Biotech Company Ltd., Taipei (TW)

(72) Inventors: Samuel CHEN, Taipei (TW); Chi-Bin LI, Taipei (TW); Ching-Ming LEE, Taipei (TW)

(21) Appl. No.: 17/337,558

(22) Filed: Jun. 3, 2021

Related U.S. Application Data
(60) Provisional application No. 63/035,063, filed on Jun. 5, 2020.

(51) Int. Cl. C12M 1/34; G01N 15/14; G01N 33/483; G06N 3/02

(52) U.S. Cl. CPC C12M 1/34; G01N 15/1434; G01N 2015/0065; G06N 3/02; G01N 2015/1486; G01N 33/4833

(57) ABSTRACT

The present invention provides a cell counting and culture interpretation method and its application, which includes: obtaining a cell culture image; segmenting the cell culture image by a cell inference model to obtain a plurality of regions corresponding to a plurality of classification parameters; calculating a culture parameter corresponding to one of the classification parameters; and determining to replace a culture medium when the culture parameter is between 0.05 and 0.15 and determining to harvest cells when the culture parameter is greater than 0.69. The present invention can provide objective and consistent standards to further improve efficiency and reduce manpower costs.

### BACKGROUND OF THE INVENTION

### 1. Field of the Invention

The disclosure is related to a method for cell counting and culture interpretation and the application thereof, and more particularly, to a method for cell counting and culture interpretation and the application thereof using a cell inference model obtained from machine learning.

### 2. Description of the Prior Art

Cell culture is the foundation of life science and clinical research. In traditional culturing process, cell culture experts observe the microscopic images of the cells, and then they can conclude the growth status of the cells based to their knowledge and experiences. They determine the actions to be taken according to the growth status, for example, to replace the culture medium or to harvest the cells. Therefore, the cultivation efficiency cannot be improved. During the mass production of cells, it would be highly educated labor-intensive if cell culture experts are asked for observing the cells one by one by naked eyes and determining the following actions. It is also difficult to objectively compare or understand the status of the cultured cells in the same batch or in different batches. In addition, a consistent interpretation standard is required for reducing the variability of human interpretation when controlling quality traceability of different batches. Therefore, objective and consistent method and system are needed for automatically calculating the number of cells and interpreting the culture status, such that it can be timely reminded to replace the culture medium or to harvest the cells at the best timing. In addition, it can record and compare cell culture status so as to serve as the basis of quality traceability.

### SUMMARY OF THE INVENTION

The disclosure provides a method for cell counting and culture interpretation, comprising: obtaining a cell culture image; segmenting the cell culture image by a cell inference model to obtain a plurality of regions corresponding to a plurality of classification parameters; calculating a culture parameter corresponding to one of the plurality of the classification parameters; and determining to replace a culture medium when the culture parameter is between 0.05 and 0.15, and determining to harvest cells when the culture parameter is greater than 0.69.

The disclosure also provides a computer readable storage medium applied in a computer and stored with instructions for executing the above method for cell counting and culture interpretation.

The disclosure further provides a system for cell counting and culture interpretation, comprising: an image capturing device, for capturing a cell culture image; and a digital interpretation unit, comprising: an input module, for obtaining the cell culture image; a cell inference model, for segmenting the cell culture image to obtain a plurality of regions corresponding to a plurality of classification parameters; a cell calculation module, for calculating a culture parameter corresponding to one of the plurality of the classification parameters; and a cell culture suggestion module, for determining to replace a culture medium when the culture parameter is between 0.05 and 0.15, and determining to harvest cells when the culture parameter is greater than 0.69.

In some embodiments, the cell inference model adopts Fully Convolutional Network (FCN) model.

In some embodiments, the plurality of the classification parameters comprises a cell parameter and a background parameter.

In some embodiments, the culture parameter is the ratio of the total area of the regions corresponding to the cell parameter to the area of the cell culture image.

In some embodiments, U-net architecture is applied to the fully convolutional network model, and the U-net architecture comprises a contracting path and an expansive path.

In some embodiments, the cell culture image is a microscopic culture image of mesenchymal stem cells, epithelial cells, endothelial cells, fibroblasts, muscle cells, osteocytes, chondrocytes, or adipocytes.

In some embodiments, the above method further comprises averaging a plurality of culture parameters if there are the plurality of culture parameters correspondingly derived from a plurality of cell culture images. The mean value of the plurality of culture parameters is used in the cell culture suggestion module.

In some embodiments, the determined range of the culture parameter is the combination with the smallest error rate among all the combinations of comparisons with expert culturing suggestions.

In some embodiments, the image capturing device is an inverted microscope with photographing functions.

In some embodiments, the system for cell counting and culture interpretation further comprises a comparison module, for creating a comparison drawing of growth curves according to different batches of the cell culture images and the culture parameters thereof corresponding to different time points.

In some embodiments, the system for cell counting and culture interpretation further comprises a storage module, for storing the cell culture image and a batch number, an initial time for culturing, a culture container, a photographing time, or an uploader information corresponding to the cell culture image.

According to the disclosure, the method and the system for cell counting and culture interpretation can automatically estimate the ratio of the area occupied by cells, and it can timely remind users to replace the culture medium or to harvest the cells at the best timing, such that the cell harvest efficiency is improved and the requirement of advanced labor is reduced. In addition, it can provide an objective and consistent standard. It is beneficial for subsequent batch traceability since each batch can be recorded and compared.

The present invention is illustrated but not limited by the following embodiments and drawings.

### BRIEF DESCRIPTION OF THE DRAWINGS

FIG. 1 is a conceptual view according to an embodiment of the disclosure.

FIG. 2 is a schematic view of the method according to an embodiment of the disclosure.

FIG. 3 is a schematic view of the system according to an embodiment of the disclosure.

FIG. 4 is a cell culture image according to an embodiment of the disclosure.

FIG. 5 is a flow chart of the method for cell counting and culture interpretation according to an embodiment of the disclosure.

FIG. 6 is a flow chart of machine learning according to an embodiment of the disclosure.

FIG. 7 is a schematic view of cell segmentation and classification according to an embodiment of the disclosure.

FIG. 8 is a curve chart of the experimental data according to an embodiment of the disclosure.

### DETAILED DESCRIPTION OF THE PREFERRED EMBODIMENT

Unless defined otherwise, all the technical and scientific terms used herein have the same meanings as are commonly understood by one of skill in the art to which this invention belongs.

As used herein, the singular form “a”, “an”, and “the” includes plural references unless indicated otherwise. For example, “an” element includes one or more elements.

As used herein, “around”, “about” or “approximately” shall generally mean within 20 percent, preferably within 10 percent, and more preferably within 5 percent of a given value or range. Numerical quantities given herein are approximations, meaning that the term “around”, “about” or “approximately” can be inferred if not expressly stated.

It will be clearly presented in the following detailed descriptions of the preferred embodiment with reference to the drawings regarding the technical content, features and effects of the disclosure.

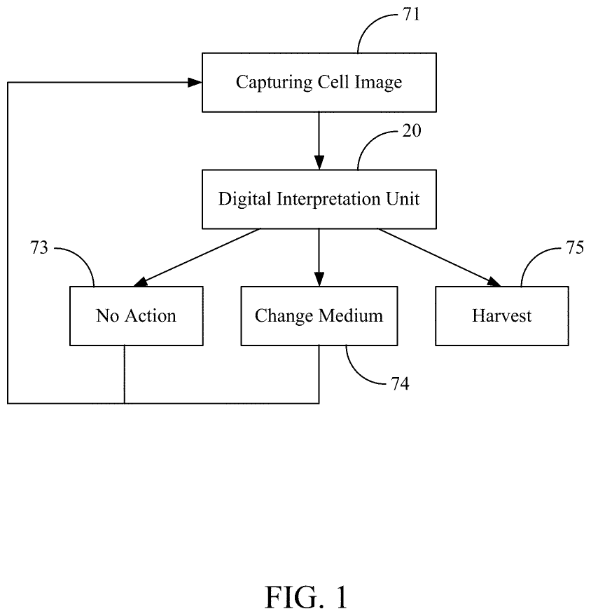

As shown in FIG. 1, in order to solve the problem that it is highly educated labor-intensive and the standards of the interpretation is not objective or consistent for determining whether to replace the culture medium or to harvest the cells by observing the culture conditions of the cells with naked eyes, the inventor came up with an innovative cell culture process. By applying the method, the digital interpretation unit 20 in the system or the computer readable storage medium according to an embodiment of the disclosure provides suggestions for cell culturing process after capturing cell images 71 at a specific time, such that the cell culture operators can proceed the cell culture based on the suggestions: no action 73, change medium 74 (or replace the culture medium), or harvest 75. Therefore, an objective and consistent standard can be provided while it saves time and labors for manual observation and interpretations.

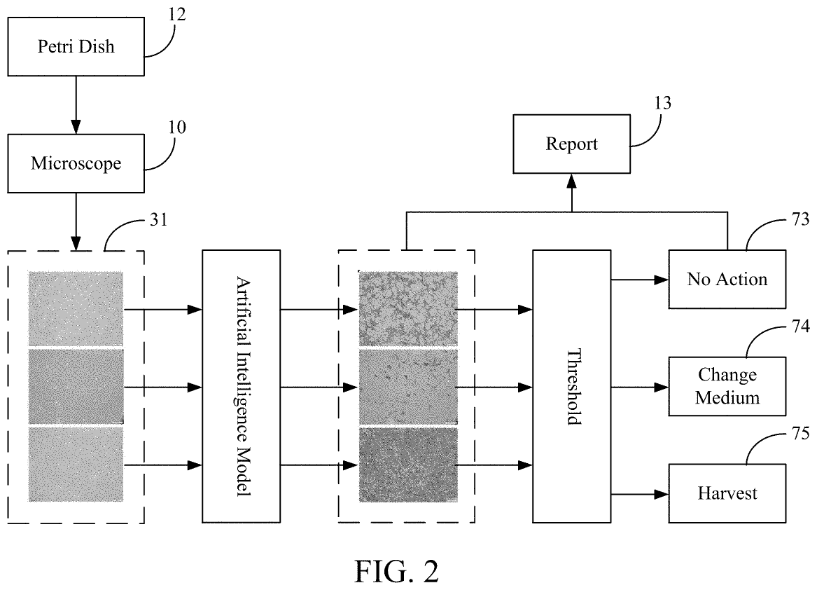

As shown in FIG. 2, in order to realize the above concept, a cell culture image 31 observed from a petri dish 12 under a microscope 10 is obtained and input to an established artificial intelligence (AI) model for analyzing the microscopic cell image from the petri dish 12. Two regions can be classified, and the dark ones are the cell regions. From the top to the bottom, after analysis, the ratio of the cell regions in each of the images is 31.42%, 9.19%, and 71.95%. The analysis result is determined by a threshold rule, such that a suggested action is provided corresponding to the analysis result: no action 73, change medium 74, or harvest 75. A report 13 is concluded according to the above information.

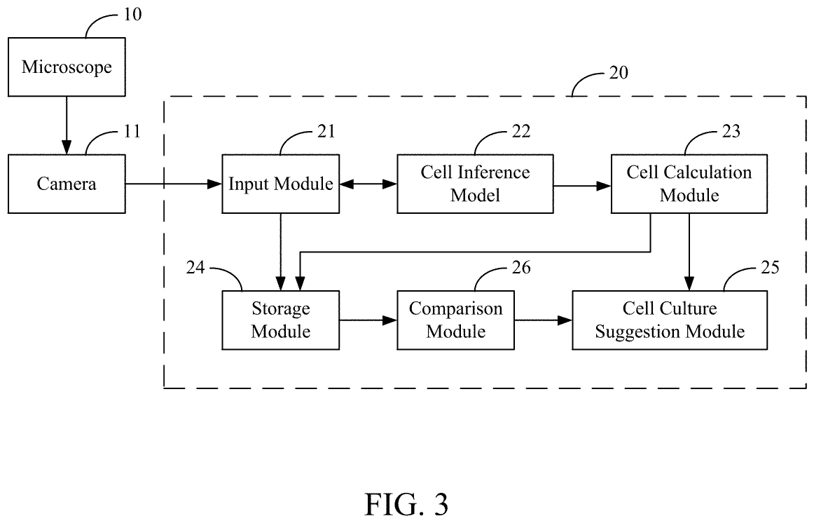

Also refer to FIG. 3, a system for cell counting and culture interpretation according to an embodiment of the disclosure comprises: an image capturing device, for obtaining a cell culture image 31; and a digital interpretation unit 20, comprising: an input module 21, for obtaining the cell culture image 31; a storage module 24, for storing the cell culture image 31; a cell inference model 22, for segmenting the cell culture image 31 to obtain a plurality of regions corresponding to a plurality of classification parameters; a cell calculation module 23, for calculating a culture parameter corresponding to one of the plurality of the classification parameters; and a cell culture suggestion module 25, for determining to replace a culture medium when the culture parameter is between 0.05 and 0.15, and determining to harvest cells when the culture parameter is greater than 0.69. The digital interpretation unit 20 further comprises a comparison module 26.

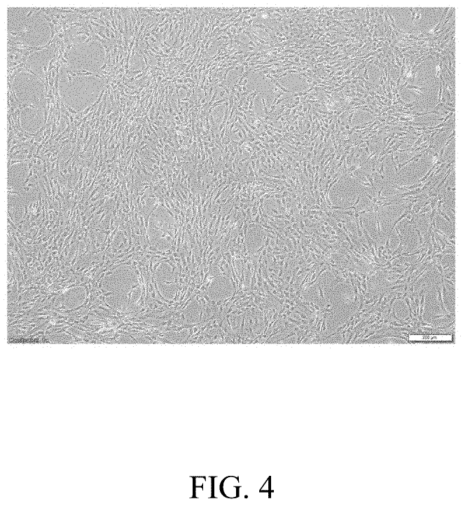

As shown in FIG. 4, culture images of mesenchymal stem cells (MSC) are used for developing the method, the digital interpretation unit 20 in the system, or the computer readable storage medium of the embodiment of the disclosure. The growth characteristic of mesenchymal stem cells is that they attach the bottom surface of the petri dish 12 and grow along the surface flatly, such that the growth curve of mesenchymal stem cells is proportional to the cell areas, and the cell growth status can be understood by analyzing the culture images. Therefore, the method, the digital interpretation unit 20 in the system, or the computer readable storage medium developed in the embodiment of the disclosure can be applied to other adherent cells, such as epithelial cells, endothelial cells, fibroblasts, muscle cells, osteocytes, chondrocytes, adipocytes and so forth. The image format of the cell culture images 31 is a JPG file, and other formats, such as PNG, GIF, BMP, and so forth can be used as well. The scale in the figure is 200 microns, and the image size is about 1360*1024 pixels, but it can be adjusted and set according to the requirements of image capturing.

First, an inverted microscope 10 is used. Light source is provided from the bottom of the petri dish 12. The cell culture image 31 is obtained by the image capturing device from the bottom of the petri dish 12. For example, the microscopic cell image is obtained from 175 Flask or CF10 by the camera 11 built in or connected with the microscope 10. Before harvesting the cells, cell culture images 31 can be captured at a fixed time every day or at specific time intervals so as to analyze and to determine if it is necessary for the cell culture operators to carry out the following processing. The area of the cell culture image is known, and the ratio of the number of cells to the area is a certain number, such that the total number of cells in the entire Petri dish 12 can be estimated by its cell area.

The digital interpretation unit 20 comprises, but not limited to, central processing units, graphic processing units, digital signal processors, or the combinations thereof used in computers, mobile communication devices, tablets, or mobile phones, or embedded microprocessors in the image capturing devices. The digital interpretation unit 20 and the image capturing device are connected through wired or wireless connection, such that the cell culture image 31 obtained by the image capturing device can be transferred to the digital interpretation unit 20. According to the embodiment of the disclosure, personal computers are used for the development, and the specifications of the computers are shown in the table below:

| | |
|---|---|
| Central Processing Unit | Intel ® Core ™ i7-7800X |
| | CPU @3.50 GHz |
| RAM (minimum specification) | 2 GB |
| Graphic Processing Unit (optional) | NVIDIA GeForce GTX 1080 Ti |
| Operation System | Linux |

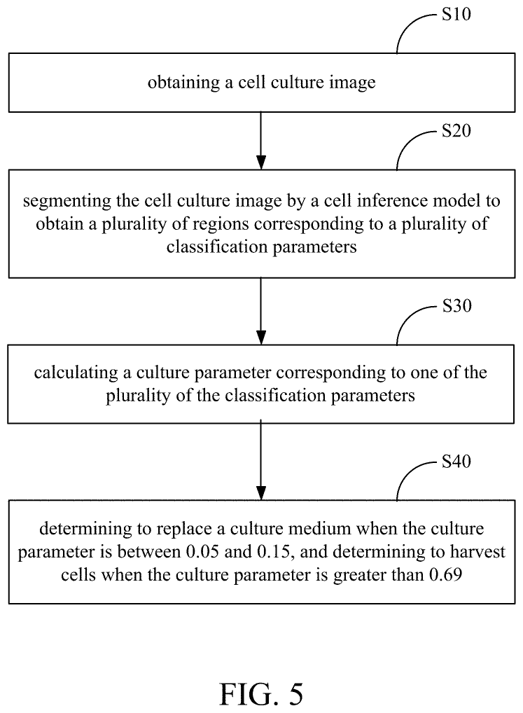

The method for cell counting and culture interpretation according to the embodiment of the disclosure is applied in the corresponding modules of the digital interpretation unit 20. Also, computer instructions of the method for cell counting and culture interpretation are stored in the computer readable storage medium according to the embodiment of the disclosure, which can execute the following method, wherein the details of each step are described in the followings. As shown in FIG. 5, the method for cell counting and culture interpretation comprises: obtaining a cell culture image 31 (step S10); segmenting the cell culture image 31 by a cell inference model 22 to obtain a plurality of regions corresponding to a plurality of classification parameters (step S20); calculating a culture parameter corresponding to one of the plurality of the classification parameters (step S30); and for determining to replace a culture medium when the culture parameter is between 0.05 and 0.15, and determining to harvest cells when the culture parameter is greater than 0.69 (step S40).

The input module 21 obtains the cell culture image 31 transferred from the image capturing device or it obtains the cell culture image 31 imported by the user (step S10). In addition, batch numbers can be established to facilitate subsequent traceability in the cell culture procedures of mass production. Therefore, the input module 21 can further obtain the information corresponding to the whole batch of the cell culture images 31, such as the batch number, the initial time for culturing, the culture container, and so forth. When the user imports a large number of cell culture images 31, the input module 21 can further obtain the information corresponding to the batch numbers, such as the entire batch of images, the shooting time, the uploader, and so forth.

Then, the storage module 24 stores the cell culture image 31 and other corresponding data transferred from the input module 21 into the storage device, or the cell inference model 22 proceeds subsequent analysis of the cell culture images. The storage device is, for example, a hard disk, a server, a memory, and so forth, which has wired or wireless connection with the digital interpretation unit 20. The storage module 24 is used for accessing the data in the storage device for subsequent analysis.

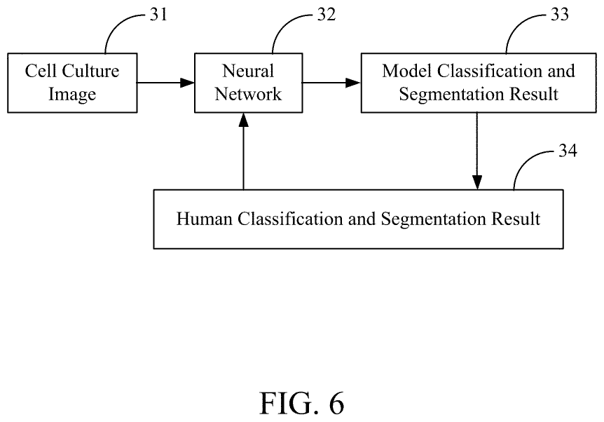

As shown in FIG. 6, supervised machine learning is applied to the cell inference model 22. The cell inference model 22 aims for solving the problem of segmentation in machine learning. A model classification and a segmentation result 33 are generated after a neural network 32 to be trained is trained by a plurality of cell culture images 31. The neural network 32 adjusts the parameters according to the differences between the model classification the segmentation result 33 and the human classification the segmentation result 34 of the cell culture experts. When a certain number of images are provided, and then with an appropriate number of adjustments, the trained neural network 32 can be used as the cell inference model 22 with its performance matching or exceeding the performance of human experts.

Therefore, if one seeks to train the cell inference model 22 for segmenting the cell culture images 31 into three categories: background N, type-A cell (for example, target cells), and type-B cell (for example, non-target cells), it is necessary to mark the target cell areas and non-target cell areas determined by the cell culture experts with their naked eye for training the machine learning model. In order to save training time, it is also possible to train the cell inference model 22 for only segmenting the cell culture images 31 into two categories: background and cell, so that only the cell areas, which are determined by the cell culture experts with their naked eye, should be marked and used for training the machine learning model.

U-net architecture of Fully Convolutional Network model (FCN) is applied to the cell inference model 22, which comprises a contracting path and an expansive path. Two convolutional layers (3×3), a rectified linear unit (ReLU) and a max pooling layer (2×2) are used in the contracting path. The number of channels is doubled for each down-sampling. A convolutional layer (2×2), a rectified linear unit (ReLU) and two convolutional layers (3×3) are used in the expansive path. Each up-sampling will also incorporate features from the corresponding down-sampling to compensate for the loss of detailed information. Finally, a convolutional layer (1×1) is used for converting the 64 channel feature vector into the required number. According to the input image, different feature maps are extracted by learning from the neighboring pixels when using pixel as a unit. Finally, an image with the same size as the original image is output, and the background areas are marked as 0 while the cell areas are marked as 1.

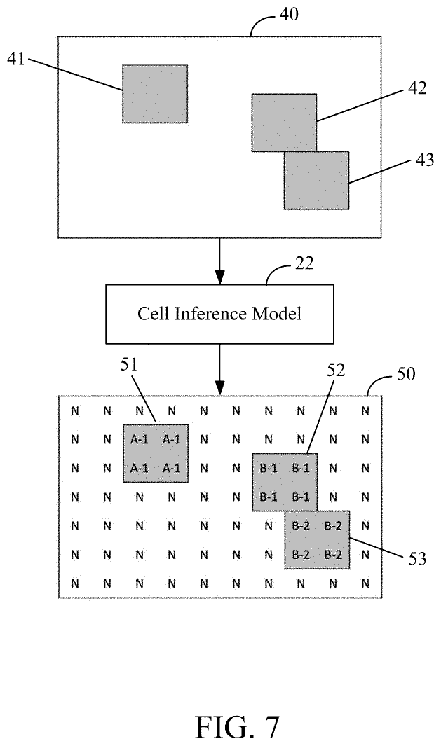

As shown in FIG. 7, the trained cell inference model 22 segments the cell culture images 31 transferred from the input module 21 into a plurality of regions and a plurality of classification parameters, such that each region corresponds to one of the classification parameters (step S20). For example, there are multiple suspected cell areas 41, 42, and 43 in the original image 40. In the processed image 50 processed by the cell inference model 22 which is trained by three classification parameters, each pixel belonging to regions 51, 52 or 53 has a cell classification and a segmentation result. The classification parameters are A, B and N. B-1 indicates the first region in classification B, B-2 indicates the second region in classification B, and A-1 indicates the first region in classification A. The regions without cells are marked as N.

If the adopted cell inference model 22 is the model for segmenting the cell culture image 31 into the background and cells, then the classification parameter comprises a cell parameter and a background parameter. The regions 51, 52 and 53 are classified as the cell region, and other regions are classified as the background region. When the classification parameters corresponding to the regions 51, 52 and 53 are determined as cell by the cell inference model 22, the area of all regions which are marked as cells according to their classification parameters can be summarized by a cell counting module 23. The culture parameter can be calculated, and the result is exported to the cell culture suggestion module 25 and stored in the storage module 24 for subsequent access. The cell counting module 23 calculates a culture parameter corresponding to one of the plurality of classification parameters (step S30). Since the culture parameter is related to the total area corresponding to the classification parameter of cell, when the total area of cell regions is confirmed, it is possible to estimate approximate number of cells, such that the culture status can be understood.

According to an embodiment of the disclosure, the culture parameter is the ratio of the total area of the regions corresponding to the cell parameter to the area of the cell culture image 31. For example, if the resolution of the cell culture image 31 is 1360×1024, there are 1360×1024=1392640 pixels in the image. If the cell inference model 22 estimates that 500000 pixels of them are the cell regions, then the ratio of the areas is 500000/1392640=35.90%, that is, the ‘culture parameter.’ For mass production, cells are cultured in a plurality of CF10, and there are 10 culture layers in each CF10. In the same batch, depending on the conditions of the culture operators, appropriate sampling can be done by obtaining a batch of the cell culture images 31. For instance, three images of each culture layer are taken along a diagonal, the culture parameters of the cell culture images 31 obtained from the same batch can be further averaged, such that the averaged culture parameter can be used for determining the subsequent actions. Thereby, according to the disclosure, ordinary laboratory personnel can easily determine the condition of cell culture and perform subsequent culture procedures.

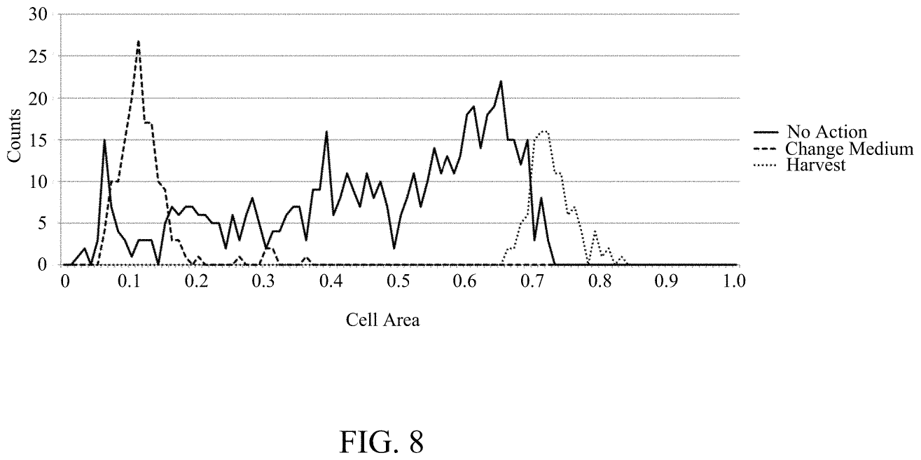

As shown in FIG. 8, 822 cell cultures images 31 are studied in order to establish rules of evaluation thresholds for automatically providing cell culture suggestions. A culture parameter (the cell region) is obtained by the cell inference model 22 from each of the cell culture images 31. Then, each of the cell culture images 31 is interpreted by cell culture experts, and a cell culture suggestion is provided and marked. The cell culture suggestions are: no action, to change medium (replace a culture medium), or to harvest. The counts of each culture suggestions under the same culture parameters (the cell region) are summarized. The horizontal axis represents the cell area and the vertical axis represents the counts. A curve of the research data for each culture suggestion is graphed.

All combinations of each culture parameters categorized into inaction, culture medium replacement or harvesting are listed. The exhaustive method is used for finding out the combination with the smallest error rate for all the three categories as compared with cell culture experts' suggestions. According to the combination with the smallest error rate, when the culture parameter is between 0.05 and 0.15, most of the cell culture images 31 are interpreted that replacing culture medium is needed by the cell culture experts and when the culture parameter is greater than 0.69, most of the cell culture images 31 are interpreted that harvesting the cell is needed by the cell culture experts (step S40). Therefore, the cell culture suggestion model 25 of the embodiment of the disclosure determines to replace a culture medium when the culture parameter is between 0.05 and 0.15, and determines to harvest cells when the culture parameter is greater than 0.69.

According to the rules studied above, the original cell culture image 31, the image processed by the cell inference model 22, the culture parameters calculated by the cell counting module 23, and the actions suggested by the cell culture suggestion module 25 can be presented in the cell culture suggestion report 13. Preferably, information, such as the batch number or batch name, number of images in the batch, initial time for culturing, photographing time or culture time (the period between the photographing time and the initial time for culturing) and other information can be presented in the cell culture suggestion report 13. Thereby, the cell culture operators need not to have a high degree of cell culture experience or knowledge, and they only need to read the report 13 regularly and follow the reminders in the report 13 to procced cell culturing procedures. In addition, the cell culture suggestion module 25 can further send a reminding message actively to the cell culture operators when a suggestion to replace the culture medium or to harvest cells is generated.

In this and some other embodiments, the digital interpretation unit 20 further comprises a comparison module 26, for creating a comparison drawing of growth curves according to different batches of the cell culture images and the culture parameters thereof corresponding to different time points. The comparison module 26 receives the batch number/batch name and the culture parameters at each time point stored in the storage module 24, and a curve of culture parameter at each time point is graphed. When presenting information from a plurality of batches numbers, the cell culture status of different batches can be compared, or the cell culture status can be compared with a standard growth curve. Therefore, quality control, growth prediction and culture adjustment can be achieved. Meanwhile, the graphical user interface can be used to obtain the corresponding information of each batch number at each time point in the curve chart, including the original images, the processed images, total number of images of the batch number/batch name, the serial number of currently displayed image, and the culture parameters.

According to the above method and the program applying the method, after studying and testing 12 images, it takes only 3.3 seconds per image for processing when applied in systems with graphics processing units, while it takes 10 seconds per image for processing when applied in systems without graphics processing units. In other words, it can save 6.7 seconds per image for calculation. Therefore, it can be understood that the graphics processing units can greatly increase the processing speed. Therefore, a large number of accurate cell culture monitoring can be provided by the method and system of the embodiment of the disclosure, and the cost of labor and time can be greatly reduced.

According to an embodiment of the disclosure, a computer readable storage medium is used in computers, phones, or tablets, and is stored with instructions for executing the above method for cell counting and culture interpretation. Users can apply the program instructions stored in the computer readable storage medium on their computers, phones, or tablets. The computer readable storage medium comprises, but not limited to, disks, optical discs, flash memories, USB devices with non-volatile memories, network storage devices, and so forth. Users can upload the cell culture images 31, which they want to analyze, to an analysis folder. Then, the program instructions are executed so as to generate a report file. Users can obtain the file report and harvest the cultured cells or to replace the culture medium according to the suggestions.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A method for cell counting and culture interpretation, comprising:
obtaining a cell culture image;
segmenting the cell culture image by a cell inference model to obtain a plurality of regions corresponding to a plurality of classification parameters;
calculating a culture parameter corresponding to one of the plurality of the classification parameters; and
determining to replace a culture medium when the culture parameter is between 0.05 and 0.15, and determining to harvest cells when the culture parameter is greater than 0.69.

2. The method according to claim 1, wherein the cell inference model adopts Fully Convolutional Network (FCN) model.

3. The method according to claim 2, wherein the plurality of the classification parameters comprises a cell parameter and a background parameter.

4. The method according to claim 3, wherein the culture parameter is the ratio of the total area of the regions corresponding to the cell parameter to the area of the cell culture image.

5. The method according to claim 2, wherein U-net architecture is applied to the fully convolutional network model, and the U-net architecture comprises a contracting path and an expansive path.

6. The method according to claim 1, wherein the cell culture image is a microscopic culture image of mesenchymal stem cells, epithelial cells, endothelial cells, fibroblasts, muscle cells, osteocytes, chondrocytes, or adipocytes.

7. The method according to claim 1, further comprising:
averaging a plurality of culture parameters if there are the plurality of culture parameters correspondingly derived from a plurality of cell culture images.

8. The method according to claim 1, wherein the determined range of the culture parameter is the combination with the smallest error rate among all the combinations of comparisons with expert culturing suggestions.

9. A system for cell counting and culture interpretation, comprising:
an image capturing device, for obtaining a cell culture image; and
a digital interpretation unit, comprising:
an input module, for obtaining the cell culture image;
a cell inference model, for segmenting the cell culture image to obtain a plurality of regions corresponding to a plurality of classification parameters;
a cell calculation module, for calculating a culture parameter corresponding to one of the plurality of the classification parameters; and
a cell culture suggestion module, for determining to replace a culture medium when the culture parameter is between 0.05 and 0.15, and determining to harvest cells when the culture parameter is greater than 0.69.

10. The system according to claim 9, wherein the digital interpretation unit further comprises a comparison module, for creating a comparison drawing of growth curves according to different batches of the cell culture images and the culture parameters thereof corresponding to different time points.

11. The system according to claim 9, wherein the digital interpretation unit further comprises a storage module, for storing the cell culture image and a batch number, an initial time for culturing, a culture container, a photographing time, or an uploader information corresponding to the cell culture image.

12. The system according to claim 9, wherein the cell inference model adopts Fully Convolutional Network (FCN) model.

13. The system according to claim 10, wherein the plurality of the classification parameters comprises a cell parameter and a background parameter.

14. The system according to claim 13, wherein the culture parameter is the ratio of the total area of the regions corresponding to the cell parameter to the area of the cell culture image.

15. The system according to claim 12, wherein U-net architecture is applied to the fully convolutional network model, and the U-net architecture comprises a contracting path and an expansive path.

16. The system according to claim 9, wherein the image capturing device is an inverted microscope with photographing functions.

17. The system according to claim 9, wherein the cell culture image is a microscopic culture image of mesenchymal stem cells, epithelial cells, endothelial cells, fibroblasts, muscle cells, osteocytes, chondrocytes, or adipocytes.

18. The system according to claim 9, wherein when there are a plurality of culture parameters correspondingly derived from a plurality of the cell culture images, a mean value of the plurality of culture parameters is used in the cell culture suggestion module.

19. The system according to claim 9, wherein the determined range of the culture parameter is the combination with the smallest error rate among all the combinations of expert suggested culturing comparisons.

20. A computer readable storage medium, applied in a computer and stored with instructions, for executing the method for cell counting and culture interpretation according to claim 1.
